(19) United States
(12) Patent Application Publication
(10) Pub. No.: US 2024/0260979 A1
(43) Pub. Date: Aug. 8, 2024

(54) Apparatus For Injection In the Skin by Superficial Incision, And Its Loading Assembly

(71) Applicant: A2M, Saint-Mesmin (FR)

(72) Inventor: Thierry Sourisseau, Saint-Mesmin (FR)

(73) Assignee: A2M, Saint-Mesmin (FR)

(21) Appl. No.: 18/580,964

(22) PCT Filed: Jul. 19, 2022
(86) PCT No.: PCT/FR2022/051434

§ 371 Date: Jan. 19, 2024

(30) Foreign Application Priority Data
Jul. 20, 2021 (FR) FR2107823

(51) Int. Cl. A61B 17/20; A61B 17/00

(52) U.S. Cl. CPC A61B 17/205; A61B 2017/00367

(57) ABSTRACT

Apparatus for injection into the skin by superficial incision comprising a main body, et tips for incision of the skin protruding from the body, the apparatus being able to have a configuration in which the tips define an entirely curved surface, the surface being convex in a longitudinal direction, and a center of curvature of a section of the surface in a longitudinal plane parallel to the longitudinal direction and the body are on the same side of the surface.

### FIELD OF THE INVENTION

The invention relates to the field of devices for injection into the skin by superficial incision, and their loading. Particularly, the invention relates to the devices for carrying out allergic diagnosis or to the devices for vaccinating by incision in the skin of a patient and to the apparatuses and methods for simultaneously carrying several devices of this type in order to simultaneously carry out several allergic diagnoses or several vaccinations.

### STATE OF THE ART

The devices for injection into the skin by superficial incision can be used for allergic diagnostic tests or vaccinations.

Diagnostic tests are medical examinations consisting in superficially penetrating a few micro-drops of a product (allergen product for example) into the upper portion of the epidermis of a patient or an animal, in order to test the reactivity to said product. These allergic skin diagnostic tests are generally called “prick tests”.

In practice, after placing a drop of product on the skin, the latter is pricked through the drop with a tip to penetrate the product under the skin, then we wait fifteen to twenty minutes before interpreting the skin reaction.

There are incision devices on the market that have a tip and whose tip contains a small volume of reactive product. Such an incision device is configured to make an incision in the skin and simultaneously penetrate the reactive product into the skin.

There are incision devices on the market containing a vaccine solution, the incision device being capable of incising the skin and simultaneously penetrating the vaccine into the skin.

Apparatuses intended to perform several tests or several vaccines simultaneously exist. Such an apparatus can be designed as a receptacle for several incision devices, that is to say it is configured to receive and carry several incision devices. Each device is for example placed so that its tip protrudes from a surface of the apparatus. The apparatus is then put into contact against the skin of the patient so as to prick the skin with each of the tips of the devices which penetrates the useful product into the skin.

However, the existing apparatuses do not allow an application of the tips so that each of the devices effectively penetrates the useful product into the epidermis. This reduces the interest of such an apparatus that does not ensure all the medical tasks (diagnostic test or vaccination) that it is supposed to fulfill simultaneously.

There is therefore a need for an apparatus that allows performing in a single application a plurality of skin incisions in order to make a useful product penetrate, and more effectively carrying out a plurality of medical tasks such as a plurality of diagnostic tests or a plurality of vaccinations.

### DISCLOSURE OF THE INVENTION

One aim of the invention is to propose an apparatus for simultaneously performing a plurality of skin incisions so as to make a useful product penetrate into the skin by each incision, thus carrying out in a single application and in a more efficient manner than in the prior art a plurality of medical tasks such as a plurality of diagnostic tests or a plurality of vaccinations.

The aim is achieved within the framework of the present invention thanks to an apparatus for injection into the skin by superficial incision comprising a main body, and tips for incision of the skin protruing from the body, the apparatus being able to have a configuration in which the tips define an entirely curved surface, the surface being convex in a longitudinal direction, and a center of curvature of a section of the surface in a longitudinal plane parallel to the longitudinal direction and the body are on the same side of the surface.

Such an arrangement of the tips makes it possible to roll the apparatus over the skin along this surface in the longitudinal direction, so that each of the tips effectively penetrates useful product into the epidermis.

The curved nature of the surface ensures the continuity of the surface and allows the absence of a ridge separating a first area from a second area of the apparatus. This allows the continuity of the application gesture: the operator not having to manage the passage from the first area to the second area, he does not have to make a particular effort in the application to produce an identical bearing from one area to another.

The apparatus is advantageously and optionally completed by the following different characteristics taken alone or in combination:
the main body comprises a face, the tips protruding from the face, the face defining when the apparatus is in the configuration an average radius of curvature in the longitudinal plane, the average radius of curvature being comprised between 7 and 20 centimeters;
two branches disposed at two longitudinal ends of the main body, the two branches being directly linked to each other when the apparatus is in the configuration so as to form a handle, the main body and the handle being on the same side of the surface;
the handle has a cylindrical shape defined by the axial direction perpendicular to the longitudinal direction;
when the apparatus is in the configuration, the surface and the face are concave in an axial direction perpendicular to the longitudinal direction, the surface being located between a center of curvature of a section of the surface in an axial plane parallel to the axial direction and the body;
the face is cylindrical, the cylindrical face being defined by an axial direction perpendicular to the longitudinal direction;
the main body comprises elements linked in pairs and juxtaposed in a plane normal to the axial direction, two of the elements each being linked to one of the branches, each connection between two adjacent elements or between an element and a branch allowing a rotation about an axis parallel to the axial direction;
each element has a flank facing each adjacent element or an adjacent branch, each branch has a flank facing an adjacent element, so that the flanks of two adjacent elements or of adjacent element and branch are complementary and, when the apparatus is in the configuration in contact with each other;
the flanks define, when the apparatus is in the configuration, a plane passing through a center of curvature of the surface and through an axis of the connection linking the two elements or the element and the branch;
devices housed in openings in the main body and carrying the respective tips, each opening passing through the main body, each element comprising one of the openings and one of the devices, the elements having an identical shape and for each pair of adjacent elements the elements having opposite directions of orientation;
each element comprises:
   a stopper configured to cover, when the apparatus is in the configuration, a head of a first device of a first adjacent element, the stopper being in contact with the first device, and/or
   an abutment wall configured, when the apparatus is in the configuration, to be in contact with a head of a second device of a second adjacent element different from the first adjacent element;
devices housed in openings of the body and carrying the respective tips, each opening passing through the main body, the configuration being an incision configuration, the apparatus being able to have a withdrawal configuration in which each opening is uncovered so as to allow a withdrawal of a device.

The invention also relates to a loading assembly comprising an apparatus as just described, and a dispenser, the dispenser comprising a housing and a strip arranged at the bottom of the housing, the housing having cavities and the strip comprising holes, the main body comprising centering reliefs of complementary shape to the cavities and disposed along lateral edges of the main body, the housing being configured to receive the main body of the apparatus so that the reliefs are inserted into the cavities, and so that the holes receive the tips.

Such an assembly is advantageously and optionally completed by the following characteristics taken alone or in combination:
the apparatus comprising devices housed in openings of the body and carrying the respective tips, each opening passing through the main body, the configuration being an incision configuration, the apparatus being able to have a withdrawal configuration in which each opening is uncovered so as to allow a withdrawal of a device, the assembly comprising a head, the head comprising channels configured to allow a sliding of an incision device, each channel passing through the head from a flared inlet in an upper face of the head up to an outlet in a lower face of the head, the head being configured to cover the main body of the apparatus when the main body in the withdrawal configuration is received in the housing, the lower face bearing on lateral walls of the dispenser, each loading channel having an outlet facing an opening so that a device sliding in the channel passes into the opening;
the channels have a helix shape around a direction of extension of the channel, so that the sliding of an incision device from the upper face up to the lower face rotates the device by an angle of 90° around the direction of extension.

Furthermore, the invention relates to a superficial skin incision method in which an apparatus carrying incision tips for allergic diagnosis is rolled over the skin of a patient by placing the tips successively in contact with the skin.

The incision method can advantageously and optionally be completed by the following characteristic: the apparatus is an apparatus as described previously, the method comprising a step of adapting the apparatus in the incision configuration before rolling it over the skin.

Finally, the invention relates to a method for preparing the apparatus of an assembly as described above, the method comprising the following steps:
cleaning the strip,
pouring a useful product into a hole of the strip,
receiving the apparatus in the housing.

The preparation method can advantageously and optionally be completed by a step of covering the main body received in the housing by the loading head and a step of inserting an incision device empty of useful product into an inlet of a loading channel.

### DESCRIPTION OF THE FIGURES

Other characteristics and advantages of the invention will still emerge from the following description, which is purely illustrative and not limiting, and should be read in relation to the appended drawings in which:

FIGS. 1, 2 and 3 schematically illustrate an apparatus for injection into the skin by superficial incision according to one embodiment of the invention,

FIG. 4 schematically illustrates a skin incision device,

FIG. 5 schematically illustrates a main body of an apparatus for injection into the skin by superficial incision according to one embodiment of the invention.

FIG. 6 schematically represents an element of the main body according to one embodiment of the invention.

FIG. 7 schematically illustrates a main body of an apparatus for injection into the skin by superficial incision according to one embodiment of the invention.

FIG. 8 schematically represents an element of the main body according to one embodiment of the invention.

FIG. 9 schematically represents a dispenser.

FIGS. 10 and 11 schematically illustrate one embodiment of a loading assembly.

### DETAILED DESCRIPTION OF THE INVENTION

Apparatus for injection into the skin

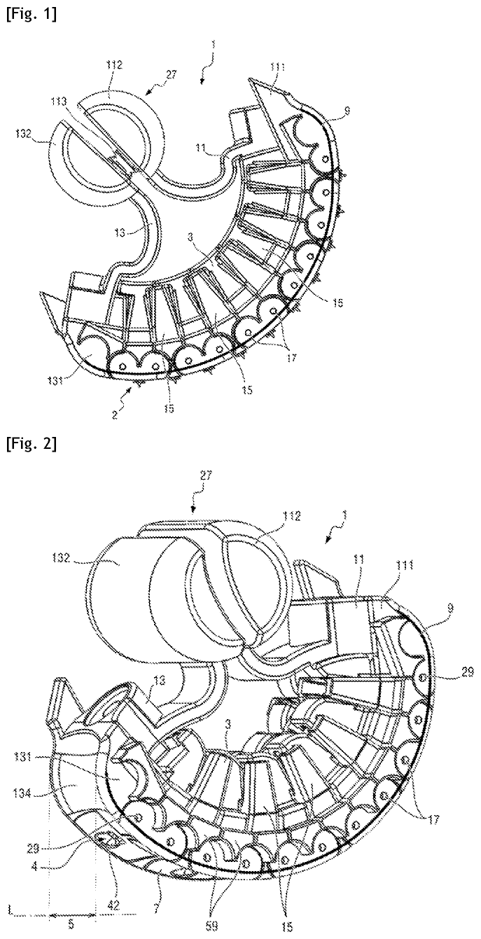

FIGS. 1, 2 and 3 schematically illustrate an apparatus according to a first embodiment of the invention.

The apparatus 1 comprises skin incision devices 2 intended to penetrate a useful product into the skin such as an allergen reagent or respectively a vaccine solution in order to test the reactivity of the person or of the animal to the reagent or respectively vaccinate the patient or the animal.

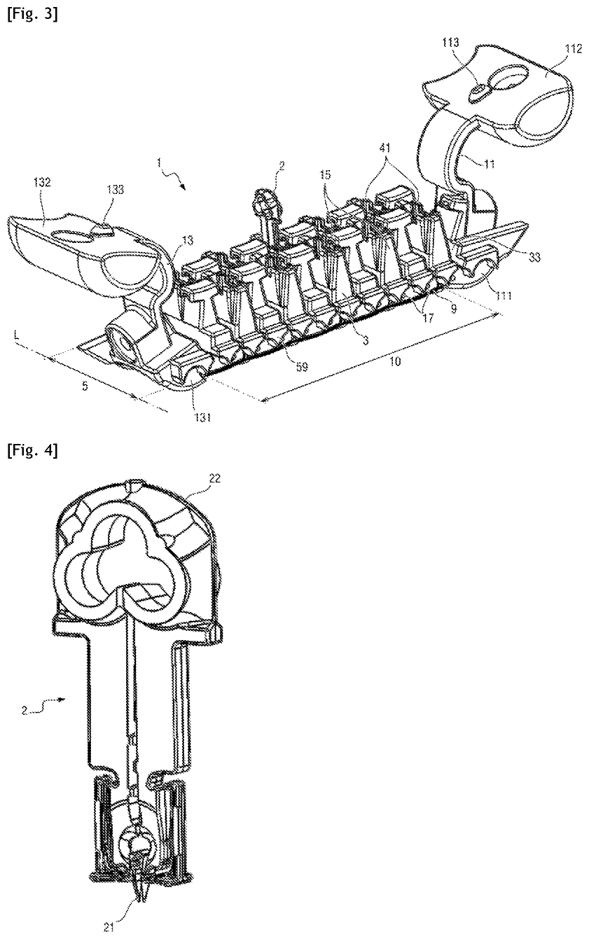

The skin incision devices 2 can in particular be the devices described in the application FR3070013. FIG. 4 schematically illustrates a skin incision device 2. Each device 2 comprises at a first end a tip 21 for incising the skin (for allergic diagnosis or for vaccination), and at a second end opposite to the first end a head 22.

The apparatus 1 comprises a main body 3 which extends over a width 5 along a first direction L called lateral direction or axial direction.

The width 5 is typically comprised between 2 and 30 cm.

The main body 3 is configured to fix skin incision devices 2.

The fixing of the devices in the main body is configured so that the tips protrude from the main body (3).

The devices can be the identical or different.

### Surfaces of the Tips in the Incision Configuration

The apparatus comprises the main body and the skin incision tips. It can be arranged in an incision configuration in which the tips define an entirely curved surface, the surface being convex in a longitudinal direction, and a center of curvature of a section of the surface in a longitudinal plane parallel to the longitudinal direction and the body are on the same side of the surface.

The incision configuration is represented in FIGS. 1 and 2.

The surface in question is virtual and passes through the ends of the tips.

This surface, called surface of the tips, is entirely curved, that is to say the surface is not planar and does not have any ridges. In other words, the surface is not a planar surface or a plurality of surfaces which join along ridges.

The surface of the tips is convex in a longitudinal direction, in other words any section of the surface of the tips in a longitudinal plane parallel to the longitudinal direction has a curvature defined by one or several centers of curvature, the center(s) of curvature being on the side of the main body. In other words, each device which has its tip on the surface is on the same side of the surface as the center(s) of curvature of the section of the surface of the tips in a longitudinal plane.

The use of the apparatus described above makes it possible to implement a superficial skin incision method in which the apparatus carrying skin incision tips 21 is rolled over the skin of a patient along the longitudinal direction, which makes it possible to put the tips successively into contact with the skin. Optionally, the surface of the tips can be concave in an axial direction perpendicular to the longitudinal direction. The surface of the tips can thus be rolled in the longitudinal direction and have a recessed shape in the axial direction. The recessed shape can in particular be adapted to the shape of a portion of the body on which the apparatus is set in motion, such as an arm or a forearm for example.

Optionally, the surface of the tips can be cylindrical, that is to say it is defined by an axis and a generating curve. The cylindrical surface is determined by straight lines parallel to the axis, the straight lines resting on the generating curve. The intersection of the defined cylindrical surface and of a plane normal to the axis is the generating curve. The surface of the tips can thus be rolled in the longitudinal direction and have a flat shape in the axial direction. This flat shape is adapted to a flat shape of a portion of the body on which the apparatus is set in motion, such as a back for example.

As the cylindrical surface is entirely curved and convex, its generating curve is entirely curved and convex.

When the surface of the tips is cylindrical, its axis is the first direction L called lateral direction or axial direction.

In the incision configuration according to this same option, the generating curve can be an arc of a circle or close to an arc of a circle.

When the generating curve is an arc of a circle, the radii of curvature of the generating curve are all equal to a predetermined value corresponding to the radius of the arc of a circle.

When the generating curve is close to an arc of a circle, the ratio of any radius of curvature of the generating curve to a predetermined value is comprised between 0.9 and 1.1.

The predetermined value of the radii of curvature can be comprised between 7 and 20 cm.

Other tip surfaces can be envisaged for the incision configuration such as spherical surfaces or parabolic surfaces.

### Face of the Main Body

The tips which protrude from the body protrude from a face 7 comprised in the main body 3.

The face 7 extends in the longitudinal plane over a finite length 10. Typically, the length 10 is comprised between 5 cm and 30 cm.

In the incision configuration, the face 7 defines an average radius of curvature in the longitudinal plane, the radius of curvature being comprised between 7 and 20 centimeters.

This face can be entirely curved, that is to say the face is not planar and has no ridges.

The face of the tips can be convex in the longitudinal direction, in other words any section of the face in a longitudinal plane has a curvature defined by one or several centers of curvature, the center(s) of curvature being on the side of the main body.

The curvature of the face in a longitudinal plane corresponds to an average radius of curvature comprised between 7 and 20 cm.

In the incision configuration, when the surface is concave in the axial direction, the face is also concave in the axial direction, the surface being located between a center of curvature of a section of the surface of the tips in an axial plane parallel to the axial direction and the body.

In the incision configuration, when the surface is cylindrical defined by the axial direction, the face is also cylindrical defined by the axial direction. The cylindrical face is generated by straight lines parallel to the axial direction L, the straight lines resting on the generating curve.

The generating curve 9 is a curved segment which is not closed on itself.

The cylindrical face 7 extends in the axial direction L over the width 5. In other words, the cylindrical face 7 is obtained by scanning the generating curve 9 over the width 5 along the axial direction L.

The generating curve 9 is contained in a plane normal to the axial direction L.

The cylindrical face 7 has the shape of a ribbon, its length 10 being greater than its width 5.

The generating curve defines a longitudinal direction in which the main body extends. The ends of the main body in this direction are called longitudinal ends.

The main body 3 extends on one side of the cylindrical face, called inner side of the cylindrical face.

The generating curve 9 can take different shapes, which imposes different shapes on the cylindrical face 7 and on the main body 3.

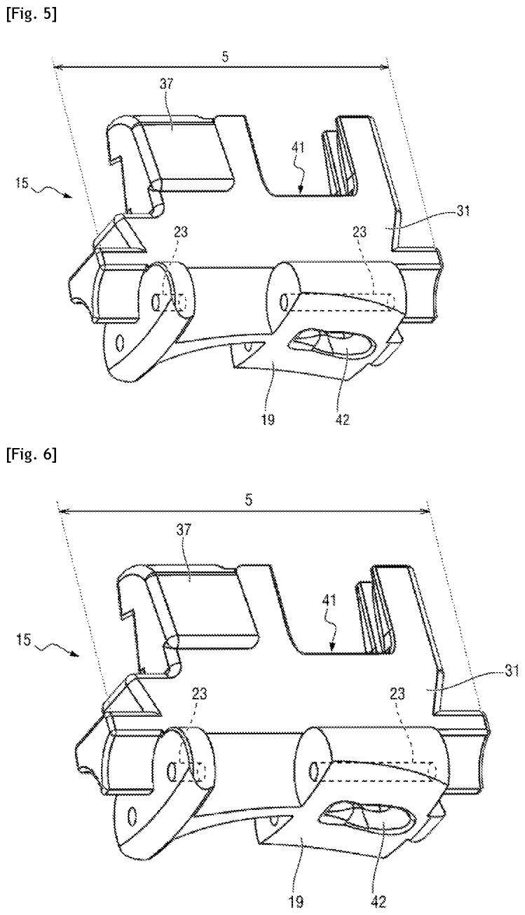

In this cylindrical face option, the opening is aligned along a direction perpendicular to the cylindrical face 7, as represented in FIG. 5 schematically illustrating a main body 3.

In the incision configuration when the surface of the tips is cylindrical along an arc of a circle or close to an arc of a circle, the cylindrical face is also an arc of a circle or close to an arc of a circle.

When the generating curve is an arc of a circle, the radii of curvature of the generating curve are all equal to a predetermined value corresponding to the radius of the arc of a circle.

When the generating curve is close to an arc of a circle, the ratio of any radius of curvature of the generating curve to a predetermined value is comprised between 0.9 and 1.1. The predetermined value corresponds in this case to an average radius of curvature of the generating curve in the normal plane.

The cylindrical face 7 extends in a plane normal to the axial direction over a length, the predetermined value (radius of the arc of a circle or average radius of curvature of the generating curve) is greater than the length.

Advantageously, the predetermined value (radius of the arc of a circle or average radius of curvature of the generating curve) is five times greater than the length. When the radii of curvature are equal to the predetermined value, the generating curve is an arc of a circle which extends over an angle comprised between 10° and 150°. The arc of a circle can preferably extend over an angle comprised between 20° and 140°.

The predetermined value (radius of the arc of a circle or average radius of curvature of the generating curve) typically takes a value between 7 cm and 20 cm. Other faces can be envisaged for the incision configuration such as spherical faces or parabolic faces.

### Branches of the Apparatus

The main body 3 comprises two branches 11, 13 disposed at two longitudinal ends of the main body 3. More specifically, the two branches 11 and 13 are placed at two ends of the generating curve.

Each branch is a rigid, non-deformable part which extends in the direction L over the width 5.

Each branch is located in the extension of the main body 3, so that in the axial direction L, the main body as well as the branches extend over the width 5.

Each branch extends, in the extension of the generating curve, from a first end 111 and 131 connected to the main body up to a second end 112 and 132.

When the apparatus comprises the two branches 11, 13, they are directly linked to each other when the apparatus is in the incision configuration. They form a handle (27), the main body (3) and the handle (27) being on the same side of the surface.

The second ends 112 and 132 comprise connecting means 113 and 133 to be linked together, so as to directly link the two branches together.

The connecting means are removable connecting means such as magnets, hook-and-loop fabric parts, fixing clips, etc.

The branches can therefore be linked together or disjointed.

When the two branches are linked together directly by their second ends, the second ends form the handle 27, that is to say the shape of the second ends linked together is easily graspable in the hand or between two fingers such as the thumb and the index finger.

Optionally, in particular, this handle 27 has a cylindrical shape defined by an axis which is the axial direction (L). In other words, the handle has a shape generated by a surface on which straight lines parallel to the axial direction (L) rest. This surface can in particular be a closed circle. Moreover, the handle may have a notch to facilitate the opening of the handle, that is to say facilitate the disengagement of the second ends.

When the radii of curvature are equal to the predetermined value, a center of the cylindrical face, the center of the arc of a circle and a center of the handle can be aligned.

The apparatus is then configured so that the handle is centered above the inner side of the cylindrical face, allowing easier and more intuitive handling of the apparatus.

Each branch 11, 13 is connected to the main body 3 so that one face, called gripping face 114 and 134, of each branch is located in the extension of the cylindrical face. The branches extend from one side of the gripping face, called inner side of the gripping face. The inner side of the gripping face is in the extension of the inner side of the cylindrical face.

Each branch is connected to the main body 3 so that each branch is connected to an element located at one end of the juxtaposition of elements. Each branch is therefore facing an element, defining a pair of adjacent branch and element. The connection between the main body and a branch is a pivot connection 29 which allows a rotation between these two parts along the axial direction L. The axis of rotation of this connection is sufficiently close to the cylindrical face and to the gripping face so that the gripping face is an extension of the cylindrical face.

In a first embodiment of the apparatus, the pivot connection between the main body and a branch is carrid out as follows: the branch comprises a passage extending in the lateral direction, a rotation shaft occupies this passage and the passage passing through an element of the main body, the element being adjacent to the branch. The shaft passing through the branch and the element links these two parts and allows a rotation therebetween around the rotation shaft.

In the second embodiment of the apparatus, the pivot connection between the main body and a branch is carried out in the following manner: a layer of material connects the branch on the one hand and the adjacent element on the other hand, the area being sufficiently fine in the directions perpendicular to the lateral direction to allow a rotation between these two parts between these two elements, around the layer.

Optionally, the two branches can have an identical shape, which limits the manufacturing costs. This option is compatible with even-numbered identical elements.

### Elements of the Main Body

The main body 3 comprises elements 15 extending over the width 5 of the main body along the lateral direction L. FIGS. 6 and 8 schematically represent an element 15 comprised in the main body, according to one particular embodiment of the apparatus.

According to this particular mode, the main body 3 comprises the cylindrical face 7 defined by an axial direction L, the tips 21 protruding from the face 7, the main body 3 comprising elements 15 linked in pairs and juxtaposed in a plane normal to the axial direction L, two of the elements 15 each being linked to a branch 11, 13, each connection 17, 29 between two adjacent elements 15 or between an element 15 and a branch 11, 13 allowing a rotation about an axis parallel to the axial direction L.

In the plane perpendicular to the lateral direction L, the elements 15 extend from the cylindrical face 7 towards the inner side. The cylindrical face comprises outer faces 19 of the elements 15.

In this same perpendicular plane, the elements 15 are juxtaposed with each other according to the generating curve 9. The adjacent elements are linked by a pivot connection 17 allowing a rotation around the lateral direction L.

The axes of these pivot connections 17 are placed close to the cylindrical face, on the inner side, so that the outer face of an element is always in contact with the outer face of an adjacent element.

The juxtaposition of the elements which are thus articulated makes it possible to confer a deformable nature to the generating curve 9 and to the main body 3.

In the first embodiment of the apparatus already mentioned above, the pivot connections 17 are all carried out in the following manner: each element 15 comprises a passage 23 extending along the lateral direction L and for each pair of adjacent elements a rotation shaft occupies a passage in each of the elements 15 of the pair, so as to pass through each element of the pair, to link these two elements and allow a rotation therebetween around the rotation shaft.

The passages in the elements are placed close to the cylindrical face, on the inner side.

In the second embodiment already mentioned above, the pivot connections are all made in the following manner: a layer of material connecting adjacent elements 15. The material layer extends in the lateral direction and forms the only connection between the elements. The layer is chosen sufficiently thin in the directions perpendicular to the lateral direction to allow a rotation around the lateral direction between these two elements around the layer. Optionally for this second embodiment, the connection can be made by several layers of materials in the extension of each other, along the lateral direction.

The layers in the elements are placed close to the cylindrical face, on the inner side.

Due to the deformable nature of the apparatus 1 produced by the different pivot connections in the main body 3 or between the main body 3 and each of the branches 11, 13, the apparatus 1 is able to have several configurations.

Particularly, the apparatus can adapt the incision configuration already presented above.

### Surfaces of the Elements and Branches in Contact

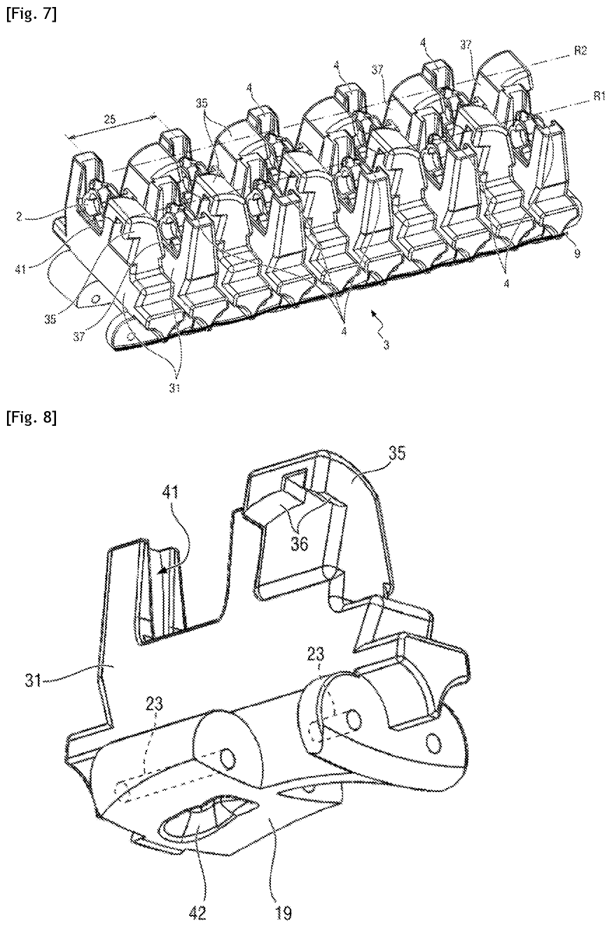

Whatever the embodiment of the pivot connections, the apparatus can, in a first option called complementary flank option, present all of the following characteristics: each element has a surface or a flank facing each adjacent element or an adjacent branch, each branch has a surface or a flank 31 facing an adjacent element, so that the surfaces or the flanks of two adjacent elements or of an element and of an adjacent branch are complementary. Such a surface is illustrated in FIGS. 6 and 7.

In this option, each element comprises two surfaces that extend over the width 5 of the element. Each surface is associated with a pivot connection of the element, so that the axis of the pivot connection is located between the outer face and the surface.

If the element is at the end of the juxtaposition of the elements, then this element comprises a surface which faces a branch and a surface which faces another element. Otherwise, each of the two surfaces of the element faces another element.

In this first option, each branch comprises a surface or a flank 33 which extends over the width 5 of the branch. The surface 33 is associated with the pivot connection of the branch with the adjacent element, so that the axis of the pivot connection is located between the gripping face and the surface. The surface of the branch faces the adjacent element of the branch.

In all cases, each surface 31, 33 faces another surface, and they are complementary to each other. The pivot connections are configured to allow the contact between two surfaces facing each other. More generally, the apparatus is configured to allow the simultaneous contact of all the pairs of surfaces facing each other.

In one particular case, the surfaces are all planar. Each surface then defines a plane passing through the axis of the pivot connection with which the surface is associated.

In the case of this first option called complementary flank option, the incision configuration corresponds to the situation where the surfaces located facing each other, as defined above, are in contact. The contact between the facing surfaces prevents one direction of the rotation of the corresponding pivot connection. Also, in the incision configuration, all the rotations are blocked in one direction. Closing the handle by linking the second ends of the branches prevents the other direction of the rotation, so that the apparatus is stable in the incision configuration. It is possible to adjust the connection between the second ends so that the apparatus has little mechanical clearance in the incision configuration. This allows stabilizing the apparatus in its incision configuration.

### Openings

The devices 2 can be fixed in the main body 3 by being housed therein, the body then comprising openings 4 to accommodate and carry the devices 2. Each opening 4 passes through the main body.

On one side of the main body, the opening comprises an inlet 41 through which a skin incision device is entered.

The opening comprises an abutment against which the skin incision device can be held.

On the other side of the main body, the opening comprises an outlet 42 through which a tip of the device extends, when the latter is bearing against the abutment.

When an incision device 2 is housed in an opening 4, the head 22 of the device protrudes from the inlet 41 of the opening and the tip 21 of the device protrudes outside the main body 3.

The openings 4 of the main body 3 can be distributed in several rows, for example two rows R1 and R2, parallel to the generating curve 9. The openings of the same row are configured so that two neighboring outlets 42 are separated by one constant pitch 25.

FIG. 7 schematically represents a main body 3 and illustrates the case where the openings are distributed along two rows R1 and R2.

The pitch 25 is typically comprised between 2 cm and 3 cm. The two rows are separated, along the lateral direction L, by a distance typically comprised between 2 cm and 3 cm. The rows are offset by approximately half a pitch, that is to say a distance twice as low as the pitch 25, from each other.

The openings 4 can all be identical, in particular if the incision devices are all identical.

Optionally, each element 15 comprises one of the openings 4 and one of the devices 2, the elements having an identical shape and for each pair of adjacent elements the elements 15 have opposite directions of orientation.

All the elements have the same shape, and two adjacent elements are disposed head to tail, the first along a first direction of the lateral direction and the second along the second direction opposite to the first direction. In this option, the element along the first direction has a shape in the region of the cylindrical face which is complementary to the shape of the adjacent element oriented along the second direction.

If the elements all have the same shape, it is possible to limit the manufacturing costs.

If, moreover, the number of elements comprised in the main body is even, it is possible to make an apparatus in which the two branches are of identical shape.

### Stopper and Abutment Wall

The apparatus can, in a second option called abutment option, have the following characteristics: each element comprises:
a stopper 35 configured to cover, when the apparatus is in the incision configuration, a head 22 of a first device 2 of a first adjacent element, the stopper 35 being in contact with the first device, and/or
an abutment wall 37 configured, when the apparatus is in the configuration, to be in contact with a head 22 of a second device of a second adjacent element different from the first adjacent element.

The first complementary flank option and the second abutment option can be implemented one without the other or be implemented in a combined manner. The combination of the two options is represented in FIGS. 6 and 7.

Each element 15 comprises an opening 4. The openings are generally distributed in two rows parallel to the generating curve 9 and separated along the lateral direction. In each row, the openings are evenly distributed along the generating curve with a constant pitch, the rows being offset by approximately half a pitch. A half pitch corresponds to the distance separating the axes of two adjacent pivot connections.

Two openings of two adjacent elements are not in the extension of each other according to the generating curve 9, they are separated by a non-zero distance along the axial or lateral direction L.

Each opening comprises, on the inner side, an inlet 41 through which a skin incision device is entered.

The element comprises a stopper 35 and an abutment wall 37 which are aligned along the generating curve with the opening of an adjacent element.

Each element comprises on the one hand an opening and on the other hand a stopper and an abutment wall separated by a non-zero distance along the lateral direction. This distance is the distance along the lateral direction separating the two parallel rows of openings.

For a given element:
its opening is aligned according to the generating curve with the stopper and the abutment wall of the adjacent element, and
its stopper and its abutment wall are aligned according to the generating curve with the opening of the adjacent element.

A stopper 35 has a recess 36 of complementary shape to a head 22 of the incision device. The shape is in particular complementary to an upper portion of the head and to a portion of the face of the head.

When an opening houses an incision device, the head of the device protrudes from the inlet of the opening.

It is possible to rotate the element carrying the device relative to an adjacent element, up to a limit arrangement where the stopper of the adjacent element covers the head of the device upon contact so that the head is housed in the recess of complementary shape. The head is then in contact with the stopper according to its upper portion and one of its faces. In this limit arrangement, the stopper can be separated by a distance of about 1 millimeter from the head of the device. In any case however; the stopper covers the head, so that the head cannot leave its opening.

An abutment wall has a planar surface which may be protruding from the element.

It is possible to rotate the element carrying the device relative to an adjacent element, up to a limit arrangement where the abutment wall of the adjacent element comes into contact with one face of the head of the device so as to press against this face. The head is then in contact with the abutment wall along one of its faces.

For a given element:
the stopper can cover only a first device housed in a first adjacent element, the stopper has an orientation according to the generating curve 9 which is the orientation of the given element towards the first adjacent element,
the abutment wall can come into contact only with a second device housed in a second adjacent element different from the first adjacent element, the abutment wall has an orientation according to the generating curve 9 different from the orientation of the stopper, namely the orientation of the given element towards the second adjacent element.

The stopper and the abutment wall are oriented along different directions of the generating curve.

When all the elements have an identical shape and the adjacent elements are disposed two by two along different directions of the lateral direction, the devices housed in the elements at the end of the juxtaposition cannot be covered by a stopper of an adjacent element or alternatively cannot be put into contact with an abutment wall of an adjacent element.

In this case, it is possible to add, as applicable, a stopper or an abutment wall to each of the branches to fulfill this role. Each branch comprises:
either a stopper having a shape complementary to a head of a skin incision device, and in a limit arrangement between a branch and an adjacent element carrying a device, the stopper of the branch covers the head of the device upon contact so that the head is housed in the recess of complementary shape,
or an abutment wall against which a head of a skin incision device can be pressed, and in a limit arrangement between a branch and an adjacent element carrying a device, the abutment wall comes into contact with one face of the head of the device.

It is thus possible to guarantee that a device, whatever the opening in which it is housed, is covered by a stopper and held against an abutment wall.

In the case of a combination of the first and second options:
the abutment wall can be parallel to a surface of the element, as defined previously,
the limit arrangement can be obtained when the surfaces of the elements facing each other come into contact,
each element 15 is configured so that the contact surfaces are located between the stopper and the abutment wall on the one hand and the outer face on the other hand.

In the case of the second stopper option, the incision configuration corresponds to the situation where each device is covered by a stopper and held against an abutment wall. The device is thus retained by the abutment in the opening, the stopper and the abutment wall. In this situation each device housed in the device is in a stable and controlled position relative to the main body.

### Withdrawal Configuration

When the apparatus comprises devices 2 housed in openings 4 of the body and carrying the respective tips, each opening 4 passing through the main body 3, the apparatus may be able to have a second configuration, called withdrawal configuration in which each opening 4 is uncovered so as to allow a withdrawal of a device 2.

When the apparatus comprises a cylindrical face and branches, the withdrawal configuration of the apparatus corresponds to a case where the cylindrical face 7 is planar and the two branches 11, 13 are disjointed.

A withdrawal configuration, when the apparatus comprises a cylindrical face 7 and branches 11, 13, is represented in FIG. 3.

The second ends of the branches are not linked directly, the removable connecting means are disengaged. The generating curve is not constrained to take the form of an arc of a circle, and takes the form of a straight segment. The cylindrical face is planar.

Relative to the incision configuration, the elements carrying the devices are rotated so that the inlets of the openings are spaced from each other. Typically, the angle of rotation between two adjacent elements making it possible to switch from the incision configuration to the withdrawal configuration is comprised between 0.5° and 35°.

As the openings pass through the main body perpendicularly to the cylindrical face, the openings of the devices are oriented parallel to each other in the withdrawal configuration.

In the withdrawal configuration, the inlets of the openings are not covered, that is to say the apparatus does not have any material in the axis of extension of the opening above the inlet of the opening, on the inner side.

The operator thus has access to the inlet in order to withdraw a device or to replace one device with another. Particularly, the apparatus is configured so that in the incision configuration the branches do not extend above the openings. In this configuration, the branches extend, from their first end, on the inner side of the cylindrical face by moving away from the center of the cylindrical face.

In the case of the first complementary flank option, this withdrawal configuration corresponds to the situation where the surfaces located facing each other, as defined above, are not in contact.

In the case of the second abutment option, this withdrawal configuration corresponds to the situation where each device is not covered by a stopper nor held against an abutment wall. The stopper of an element is therefore configured to cover a device of an adjacent element in the incision configuration and in addition so that the rotation with the adjacent element which allows switching to the withdrawal configuration is sufficient so that the stopper no longer covers the device.

### Use of the Apparatus for Skin Application

The apparatus 1 as described above comprises a plurality of devices which can be prepared so that they each contain a particular allergen reagent or a particular vaccine solution.

Such a structure in its incision configuration has the tips of the prepared devices which are oriented radially outwards along a surface which is convex in a longitudinal direction.

An operator can then apply the tips on the skin of a person or an animal by rolling the apparatus in the longitudinal direction. By doing so, the tips approach the skin, prick the epidermis then move away from the skin. The convex nature ensures that the application of the tips is performed under the same conditions from one tip to another. The homogeneous and uniform application of the different tips allows each tip to effectively incise the skin and inject the useful product into the skin.

If the handle has a cylindrical shape which extends along the lateral direction, the application of the apparatus is easier for the operator. Holding the handle between the thumb and the index finger makes it possible to use the rotation of the wrist to roll the apparatus over the patient's skin.

### Dispenser

As a complement to the apparatus, a loading assembly of this structure is proposed.

In the first embodiment of a loading assembly, the latter comprises an apparatus as presented previously and a dispenser 50.

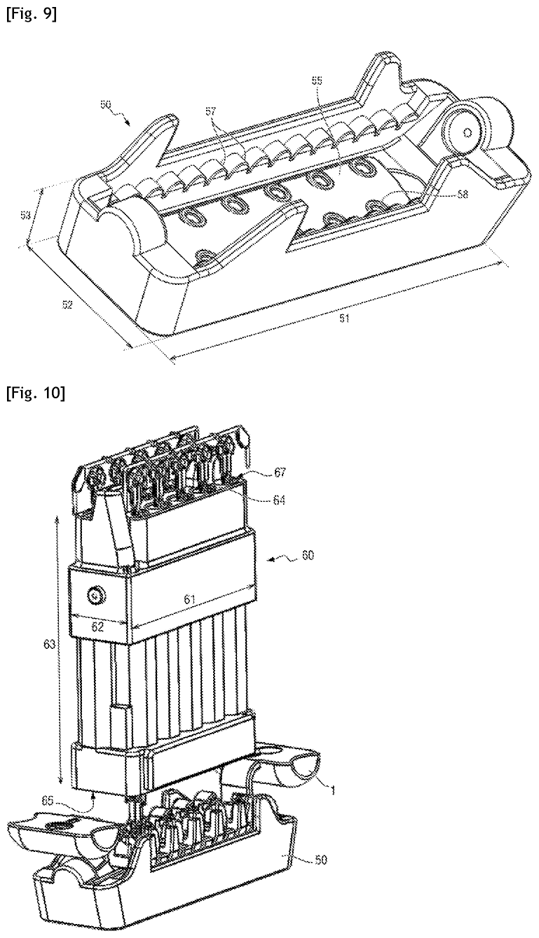

FIG. 9 schematically represents a dispenser 50.

The dispenser 50 is a rigid, non-deformable part which essentially has the shape of a cuboid or rectangular parallelepiped. The dispenser has a length 51, a width 52 and a height 53, the length 51 being significantly greater than the width 52 and the height 53.

The length of the dispenser is greater than the length of the generating curve of the main body.

The width of the dispenser is greater than the width 5 of the main body.

The dispenser comprises a housing in an upper face of the dispenser.

The upper face is defined by a length and a width.

The housing is centered in the upper face and defines a volume in the dispenser which has essentially the shape of a cuboid or rectangular parallelepiped which is smaller in each of the three dimensions than the dispenser itself.

The housing has a length parallel to the length of the dispenser which is significantly greater than its width and its height, respectively parallel to the width and height of the dispenser.

The length of the housing is greater than the length of the generating curve of the main body.

The width of the housing is barely greater than the width 5 of the main body.

The housing has a bottom parallel to the upper face.

On the sides of the housing defined by a length and a height of the housing, the housing comprises centering cavities 57.

The main body comprises centering reliefs 59 of complementary shape to the centering cavities 57, the centering reliefs being disposed along lateral edges of the main body, that is to say along the edges at both ends of the width of the main body.

The dispenser has, at the bottom of the housing, a recess which is also centered relative to the upper face and which also has the shape essentially of a cuboid or rectangular parallelepiped. The recess is smaller in each of the three dimensions than the housing.

The recess defines a bottom parallel to the upper face.

The housing does not pass right through the dispenser.

The dispenser is configured to be laid on a lower face opposite to the upper face, so that the housing is open upwards.

The height of the dispenser is therefore oriented along the vertical direction.

The recess is occupied by a strip 55, for example a glass strip or a plastic strip, for example a plastic comprising Cyclo Olefin (Co)-Polymers.

The strip is a plate with a thickness comprised between 2 and 8 millimeters.

The strip can be removed and then freey placed back in the recess. Notches in the dispenser can be provided on the edges of the recess in order to facilitate the placement or withdrawal of the strip in the recess.

The strip comprises holes 58, the number of holes is equal to the number of openings comprised in the main body. The arrangement of the holes corresponds to the arrangement of the openings of the main body when it is in the withdrawal configuration.

The housing is configured to receive the main body of the apparatus in a withdrawal configuration.

In the withdrawal configuration, the main body has a straight generating curve and a planar cylindrical face.

The length of the housing and the width of the housing are adjusted to allow the insertion of the main body therein so that:
the planar cylindrical face is inserted into the housing and faces, or comes into contact with, the strip, and
the centering reliefs are inserted into the centering cavities.

The adjustment of the housing is such that the centering reliefs can be inserted at the bottom of the centering cavities.

The strip holes are positioned to receive, when the apparatus is inserted into the dispenser, the tips of the devices protruding from the planar cylindrical face.

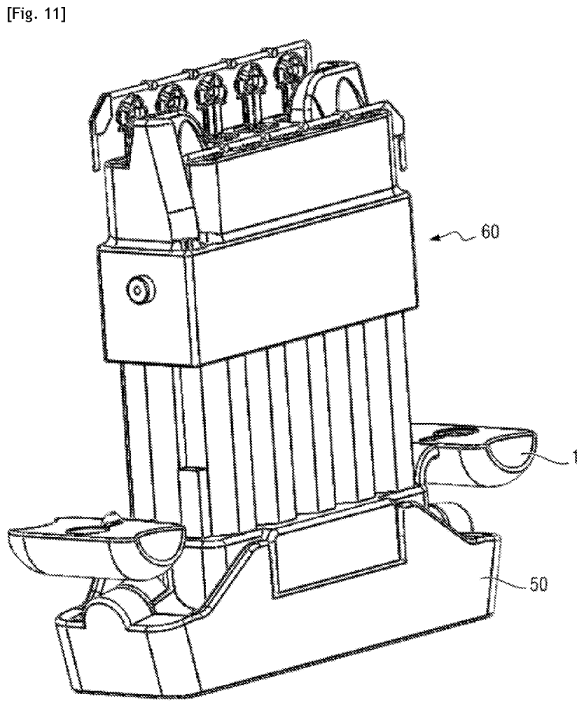

FIG. 10 schematically represents an apparatus 1 in withdrawal configuration received in a dispenser 50.

The dimensions of the housing are adjusted to the dimensions of the main body but also to the dimensions of the branches. In the withdrawal configuration, the first ends of the branches are in the extension of the main body. A portion of the branches, on the side of their first ends, is inserted into the housing at the same time as the main body.

The dimensions of the housing are adjusted to reduce the mechanical clearance when the apparatus is inserted into the housing. This makes it possible to stabilize the apparatus in the dispenser.

For this purpose, the housing has a sufficiently high height so that all of the pivot connections of the apparatus are inside the volume defined by the housing when the apparatus is inserted into the dispenser.

In addition, it is possible to provide for one or two centering reliefs per element.

By inserting the apparatus into the dispenser, this allows stabilizing the position of each pivot connection.

The insertion can be made easier by adapting the shape of the housing and of the apparatus. For example, the housing may have edges flared outwards, that is to say towards the upper face and correspondingly the main body and the branches may have flared edges.

The shape of the centering relief can be adapted to the presence of the pivot connections. For example, the relief can present two curved surfaces facing the two axes of the pivot connections of the element. Each curved surface defines one or several centers of curvatures which are all on the side of the relief where the axis of the pivot connection is located. The pivot cavity has the complementary shape to the centering relief.

The movements of the centering relief relative to the pivot connection induce a movement against the cavity generally in the direction of the curved surface, and do not oppose the insertion of the relief into the cavity. The insertion of the apparatus into the dispenser is thus facilitated, and moreover the position of each pivot connection is stabilized.

The apparatus and the dispenser may optionally comprise removable connecting means to be linked together.

The connecting means are removable connecting means such as magnets, hook-and-loop fabric parts, fixing clips, etc.

For example, the branches can comprise removable connecting means on the gripping face and the dispenser in the corresponding position in the housing comprises complementary connecting means.

This allows, by inserting the apparatus into the dispenser, stabilizing the position of each pivot link.

### Use of the Dispenser to Load the Apparatus

In one embodiment, the skin incision devices can be reloaded into the apparatus using a dispenser as presented above.

In a first step, the strip is prepared by being cleaned, then by pouring at least one useful product (an allergen reagent or a vaccine solution) into at least one hole of the strip.

It is obviously possible to pour a useful product into several holes or even into all the holes.

The useful products poured may be the identical or different from one hole to another.

The slide can be placed in the recess before or after pouring the useful products.

The apparatus is suitable in the withdrawal configuration.

If it comprises empty incision devices, that is to say not loaded with a useful product, then it is not useful to replace them.

If it comprises already used incision devices, then they can be withdrawn from the apparatus and replaced with empty incision devices. For this, it is possible, before pouring a useful product into several holes or even into all the holes, to house the apparatus in the withdrawal configuration to withdraw the already used incision devices therefrom and replace them with empty devices.

In both cases, an apparatus in the withdrawal configuration comprising incision devices empty of useful product are obtained.

The apparatus is then placed in the housing. The tips of the incision devices are thus received in the holes in the strip filled with useful product. The tips of the devices put into contact with the useful product draw an amount of it. The incision devices are thus each loaded with a useful product.

In a final step, the apparatus is adapted into an incision configuration and ready to be used for an application on the skin of a person or an animal.

### Loading Head

In a second embodiment of the assembly, the latter comprises, in addition relative to the first embodiment, a loading head.

FIGS. 10 and 11 schematically illustrate the second embodiment.

The loading head 60 is a rigid, non-deformable part which generally has the shape of a cuboid or rectangular parallelepiped.

The loading head has a length 61, a width 62 and a height 63, the height 63 and the length 61 being significantly greater than the width 62.

The length and the height of the loading head have approximately the same value which corresponds to the length of the generating curve of the main body.

The width of the loading head is approximately equal to the width of the dispenser.

The loading head is configured to be laid on the dispenser such that the length, the width and the height of the loading head are measured in directions respectively parallel to those where the length, the width and the height of the dispenser are measured.

The loading head comprises a lower face 65 defined by a length and a width configured to cover the dispenser, and an upper face 64 opposite this lower face. The loading head is configured to cover upon contact the main body of the apparatus in the withdrawal configuration when it is received in the housing, the lower face bearing on lateral walls of the dispenser, that is to say walls of the dispenser defined by a height and a length of the dispenser.

The height of the loading head is therefore oriented along the vertical direction. The lower face of the loading head may have a relief adapted to a relief of the main body on the inner side in the withdrawal configuration. In the case of the first and/or of the second option, the relief of the main body on the inner side can in particular be defined by the facing surfaces and/or the stoppers and the abutment walls. In this case, the inner face of the loading head is hollowed out with reliefs so as not to be blocked by these elements.

The loading head can optionally comprise shoulders protruding from lateral edges of the loading head, on the inner face side, the lateral edges being defined by a length and a height of the loading head. These shoulders are configured to bear on lateral walls of the dispenser, when the loading head covers the main body of the apparatus in the withdrawal configuration when it is received in the housing.

In a horizontal plane, the position of the loading head can be fixed by complementarity of the relief of the inner face of the head with the relief of the main body on the inner side in the withdrawal configuration.

The loading head comprises loading channels configured to allow the sliding of an incision device. More specifically, a device can slide into the channel when it is inserted into the channel in the direction defined by the head and tip of the device, the tip being inserted first.

Each channel passes through the loading head from the upper face 64 to the lower face 65.

Each channel includes a flared inlet 67 on the upper face side and an outlet on the lower face side.

The flared inlet allows for simple insertion of a device. Particularly, an operator can grasp a device through its head, place the tip inside the channel through the inlet of the channel and then release the head of the device to slide it into the channel.

When the loading head covers the main body received in the housing, each loading channel outlet is located facing an opening of the main body. If a device slides into the channel, it exits from the loading head and enters the opening in correspondence of the channel.

Optionally, the loading channels have a helix shape around a direction of extension of the channel—from the lower face to the upper face—so that the sliding of an incision device from an upper face to a lower face rotates the device by an angle comprised between 5 and 90° around the direction of extension. This option makes it possible to adjust the arrangement of the device relative to the main body. Particularly, if the devices are packaged in a group of devices arranged in a row, the flared inlets can correspond to this packaging and the helix shape of the channels allows an arrangement of the devices installed in the main body, different from the arrangement of the packaging.

### Use of the Loading Head to Load the Apparatus

In one embodiment presented above, the incision devices 2 can be loaded or reloaded in the apparatus using a dispenser. It is possible to complete this method with a step of covering the main body received in the housing by the loading head.

This coverage step occurs if the apparatus comprises already used incision devices and if an operator has withdrawn them from the apparatus.

Before replacing them with empty incision devices, the main body received in the housing is covered by the loading head.

A step of inserting an incision device empty of useful product into an inlet of a loading channel is then carried out. This step can be repeated for multiple incision devices in multiple loading channels. The apparatus thus receives a plurality of incision devices empty of useful product. Each device tip is then in contact with the useful product present in a hole of the strip and is loaded with useful product.

The loading head can be withdrawn above the apparatus, the apparatus can be withdrawn from the dispenser and arranged according to the incision configuration.

1. An apparatus for injection into the skin by superficial incision comprising:
a main body;
two branches disposed at two longitudinal ends of the main body; and
tips for incision of the skin, the tips protruding from the main body, the tips comprising ends;
the apparatus being able to have a configuration in which
the ends are located on an entirely curved surface, the surface being convex in a longitudinal direction,
a center of curvature of the surface in a longitudinal plane parallel to the longitudinal direction and the main body are located on a side of the surface, and
the two branches are directly linked to each other so as to form a handle, the main body and the handle being on the side of the surface.

2. The apparatus according to claim 1 wherein the main body comprises a face, the tips protruding from the face, the face being bent, when the apparatus is in the configuration, an average radius of curvature of the face in the longitudinal plane being comprised between 7 and 20 centimeters.

3. The apparatus according to claim 1 wherein the handle has a cylindrical shape defined by an axial direction (L) perpendicular to the longitudinal direction.

4. The apparatus according to claim 2, wherein, when the apparatus is in the configuration, the surface and the face are concave in an axial direction perpendicular to the longitudinal direction, the surface being located between the main body and a center of curvature of a section of the surface in an axial plane parallel to the axial direction.

5. The apparatus according to claim 2, wherein the face is cylindrical, the cylindrical face being defined by an axial direction (L) perpendicular to the longitudinal direction.

6. The apparatus according to claim 5, wherein the main body comprises elements linked in pairs, the elements being juxtaposed in a plane normal to the axial direction (L), two of the elements being linked to one of the branches, the apparatus being configured so that two adjacent elements are linked with a connexion allowing a rotation about an axis parallel to the axial direction (L), the apparatus being configured so that each of the two elements linked to one of the branches is linked to the one of the branches with a connexion allowing a rotation about an axis parallel to the axial direction (L).

7. The apparatus according to claim 6, wherein each element has an element flank, the element flanck facing an adjacent element or an adjacent branch, each branch having an branch flank, the branch flanck facing an adjacent element, the element flanks of two adjacent being complementary and, when the apparatus is in the configuration the element flanks of two adjacent being in contact with each other, a branch flank and an element flank of an adjacent element being complementary and, when the apparatus is in the configuration, the branch flank and the element flank of the adjacent element are in contact with each other.

8. The apparatus according to claim 7 wherein the element flanks of two adjacent define, when the apparatus is in the configuration, a plane passing through a center of curvature of the surface and through an axis of the connection linking the two elements, wherein the branch flank and the element flank of the adjacent element define, when the apparatus is in the configuration, a plane passing through a center of curvature of the surface and through an axis of the connection linking the branch and the adjacent element.

9. The apparatus according to claim 6 comprising devices housed in openings of the main body, each device being configured to carry a tip, each opening passing through the main body, each element comprising one of the openings and one of the devices, the elements having an identical shape and for each pair of adjacent elements the elements having opposite directions of orientation.

10. The apparatus according to claim 9, wherein each element comprises:
a stopper configured to cover, when the apparatus is in the configuration, a head of a first device of a first adjacent element, the stopper being in contact with the first device, or
an abutment wall configured, when the apparatus is in the configuration, to be in contact with a head of a second device of a second adjacent element, or
a stopper and an abutment wall, the stopper being configured to cover, when the apparatus is in the configuration, a head of a first device of a first adjacent element, the stopper being in contact with the first device, the abutment wall being configured, when the apparatus is in the configuration, to be in contact with a head of a second device of a second adjacent element different from the first adjacent element.

11. The apparatus according to claim 1 comprising devices housed in openings of the main body, each device being configured to carry a tip, each opening passing through the main body, the configuration being an incision configuration, the apparatus being able to have a withdrawal configuration in which each opening is uncovered so as to allow withdrawal of a device.

12. A loading assembly comprising an apparatus according to claim 1, and a dispenser, the dispenser comprising a housing and a strip arranged at the bottom of the housing, the housing comprising cavities and the strip comprising holes, the main body comprising centering reliefs, each centering relief having a complementary shape to a shape of a cavity, the centering reliefs being disposed along lateral edges of the main body, the housing being configured to receive the main body of the apparatus so that the centering reliefs are inserted into the cavities, and so that the holes receive the tips.

13. A loading assembly comprising an apparatus according to claim 10, and a dispenser, the dispenser comprising a housing and a strip arranged at the bottom of the housing, the housing comprising cavities and the strip comprising holes, the main body comprising centering reliefs, each centering relief having a complementary shape to a shape of a cavity, the centering reliefs being disposed along lateral edges of the main body, the housing being configured to receive the main body of the apparatus so that the centering reliefs are inserted into the cavities, and so that the holes receive the tip the apparatus comprising devices housed in openings of the main body, each device being configured to carry a tip, each opening passing through the main body, the configuration being an incision configuration, the apparatus being able to have a withdrawal configuration in which each opening is uncovered so as to allow withdrawal of a device, the assembly comprising a head, the head comprising channels configured to allow a sliding of a device, each channel passing through the head from a flared inlet in an upper face of the head up to an outlet in a lower face of the head, the head being configured to cover the main body of the apparatus when the main body in withdrawal configuration is received in the housing, the lower face bearing on lateral walls of the dispenser, each loading channel having an outlet facing an opening so that when a device slides in the channel, the device passes into the opening.

14. The assembly according to claim 13 wherein the channels have a helix shape around a direction of extension of the channel, so that the sliding of an incision device from the upper face up to the lower face rotates the device by an angle of 90° around the direction of extension.

15. A superficial skin incision method, the method comprising:
a step of rolling an apparatus carrying skin incision tips over the skin of a patient, the step of rolling comprising placing ends of the tips successively in contact with the skin, the ends being located on an entirely curved surface, the surface being convex in a longitudinal direction, a center of curvature of the surface in a longitudinal plane parallel to the longitudinal direction and a main body of the apparatus being located on a side of the surface.

16. A method for preparing the apparatus of an assembly according to claim 12, the process comprising the following steps:
cleaning the strip,
pouring a liquid product into a hole of the strip,
receiving the apparatus in the housing.

17. A method for preparing the apparatus of an assembly according to claim 13, the process comprising the following steps:
cleaning the strip,
pouring a liquid product into a hole of the strip,
receiving the apparatus in the housing,
covering the main body received in the housing by the loading head and a step of inserting an incision device empty of liquid product into an inlet of a loading channel.
